(19) United States
(12) Patent Application Publication
(10) Pub. No.: US 2009/0196900 A1
(43) Pub. Date: Aug. 6, 2009

(54) Use of Phosphodiesterase Inhibitor as a Component of Implantable Medical Devices

(75) Inventor: Robert J. Melder, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/024,167

(22) Filed: Feb. 1, 2008

(51) Int. Cl. A61F 2/04; A61K 31/519; A61K 31/4985

(52) U.S. Cl. 424/423; 514/262.1; 514/250

(57) ABSTRACT

Implantable medical devices having coatings comprising phosphodiesterase inhibitors are disclosed. Specifically, coatings comprising phosphodiesterase-5 inhibitors are disclosed. The phosphodiesterase-5 inhibitors include sildenafil, tadalafil, vardenafil or pharmaceutically acceptable derivatives thereof. The medical devices can include stents, catheters, micro-particles, probes and grafts

### FIELD OF THE INVENTION

The present invention relates to medical devices to improve the vascular or platelet response to nitric oxide. Specifically, the present disclosure relates to stents that provide in situ controlled release of phosphodiesterase inhibitors. More specifically, the present disclosure provides vascular stents that provide phosphodiesterase-5 inhibitors to tissue in need of nitric oxide mediated vasodilatation.

### BACKGROUND OF THE INVENTION

Nitric oxide (NO) is a simple diatomic molecule that plays a diverse and complex role in cellular physiology. Less than 25 years ago NO was primarily considered a smog component formed during the combustion of fossil fuels mixed with air. However, as a result of the pioneering work of Ferid Murad et al. it is now known that NO is a powerful signaling compound and cytotoxic/cytostatic agent found in nearly every tissue including endothelial cells, neural cells and macrophages. Mammalian cells synthesize NO using a two step enzymatic process that oxidizes L-arginine to N-ω-hydroxy-L-arginine, which is then converted into L-citrulline and an uncharged NO free radical. Three different nitric oxide synthase enzymes regulate NO production. Neuronal nitric oxide synthase (NOSI, or nNOS) is formed within neuronal tissue and plays an essential role in neurotransmission; endothelial nitric oxide synthase (NOS3 or eNOS), is secreted by endothelial cells and induces vasodilatation; inducible nitric oxide synthase (NOS2 or iNOS) is principally found in macrophages, hepatocytes and chondrocytes and is associated with immune cytotoxicity.

Neuronal NOS and eNOS are constitutive enzymes that regulate the rapid, short-term release of small amounts of NO. These minute amounts of NO activate guanylate cyclase which elevates cyclic guanosine monophosphate (cGMP) concentrations which in turn increase intracellular Ca⁺² levels. Increased intracellular Ca⁺² concentrations result in smooth muscle relaxation which accounts for NO's vasodilating effects. Inducible NOS is responsible for the sustained release of larger amounts of NO and is activated by extracellular factors including endotoxins and cytokines. These higher NO levels play a key role in cellular immunity.

Medical research is rapidly discovering therapeutic applications for NO including the fields of vascular surgery and interventional cardiology. Procedures used to clear blocked arteries such as percutaneous transluminal coronary angioplasty (PTCA) (also known as balloon angioplasty) and atherectomy and/or stent placement can result in vessel wall injury at the site of balloon expansion or stent deployment. In response to this injury a complex multi-factorial process known as restenosis can occur whereby the previously opened vessel lumen narrows and becomes re-occluded. Restenosis is initiated when thrombocytes (platelets) and inflammatory cells migrating to the injury site release cytokines and growth factors into the injured endothelium. Thrombocytes begin to aggregate and adhere to the injury site initiating thrombogenesis, or clot formation. As a result, the previously opened lumen begins to narrow as thrombocytes and fibrin collect on the vessel wall. In a more frequently encountered mechanism of restenosis, the cytokines and growth factors released by activated thrombocytes and inflammatory cells adhering to the vessel wall stimulate over-proliferation of vascular smooth muscle cells during the healing process, restricting or occluding the injured vessel lumen. The resulting neointimal hyperplasia is the major cause of stent restenosis.

Recently, NO has been shown to significantly reduce thrombocyte aggregation and adhesion as well as inhibit inflammation; this combined with NO's cytostatic properties may significantly reduce vascular smooth muscle cell proliferation and help prevent restenosis. Thrombocyte aggregation occurs within minutes following the initial vascular insult and once the cascade of events leading to restenosis is initiated, irreparable damage can result. Moreover, the risk of thrombogenesis and restenosis persists until the endothelium lining the vessel lumen has been repaired. Therefore, it is essential that NO, or any anti-restenotic agent, reach the injury site immediately.

One approach for providing a therapeutic level of NO at an injury site is to increase systemic NO levels prophylactically. This can be accomplished by stimulating endogenous NO production or using exogenous NO sources. Methods to regulate endogenous NO release have primarily focused on activation of synthetic pathways using excess amounts of NO precursors like L-arginine, or increasing expression of nitric oxide synthase (NOS) using gene therapy. U.S. Pat. Nos. 5,945,452, 5,891,459 and 5,428,070 describe sustained NO elevation using orally administrated L-arginine and/or L-lysine. However, these methods have not been proven effective in preventing restenosis. Regulating endogenously expressed NO using gene therapy techniques remains highly experimental and has not yet proven safe and effective. U.S. Pat. Nos. 5,268,465, 5,468,630 and 5,658,565, describe various gene therapy approaches.

Exogenous NO sources such as pure NO gas are highly toxic, short-lived and relatively insoluble in physiological fluids. Consequently, systemic exogenous NO delivery is generally accomplished using organic nitrate prodrugs such as nitroglycerin tablets, intravenous suspensions, sprays and transdermal patches. The human body rapidly converts nitroglycerin into NO; however, enzyme levels and co-factors required to activate the prodrug are rapidly depleted, resulting in drug tolerance. Moreover, systemic NO administration can have devastating side effects including hypotension and free radical cell damage. Therefore, using organic nitrate prodrugs to maintain systemic anti-restenotic therapeutic blood levels is not currently possible.

Therefore, considerable attention has been focused on localized, or site specific, NO delivery to ameliorate the disadvantages associated with systemic prophylaxis. Implantable medical devices and/or local gene therapy techniques including medical devices coated with NO-releasing compounds, or vectors that deliver NOS genes to target cells, have been evaluated. Like their systemic counterparts, gene therapy techniques for the localized NO delivery have not been proven safe and effective. There are still significant technical hurdles and safety concerns that must be overcome before site-specific NOS gene delivery will become a reality.

Many anti-restinotic compounds can be toxic when administered systemically in large amounts. Furthermore, the exact cellular functions that must be inhibited and the duration of inhibition needed to achieve prolonged vascular patency (greater than six months) is not presently known. Moreover, it is believed that each drug may require its own treatment duration and delivery rate. Therefore, in situ, or site-specific drug delivery using anti-restenotic coated stents has become the focus of intense clinical investigation.

Recent human clinical studies on stent-based anti-restenotic delivery have centered on rapamycin and paclitaxel. In both cases excellent short-term anti-restenotic effectiveness has been demonstrated. However, side effects including stent malapposition, potentially due to cell loss and vascular remodeling have also been seen. The resulting vascular pathology can lead to stent instability and increased risk of late stent thrombosis. Furthermore, the extent of cellular inhibition may be so extensive that normal re-endothelialization will be significantly delayed. The endothelial lining is essential for maintaining normal vascular function since it is an endogenous source of nitric oxide through the production of endothelial nitric oxide synthase. Therefore, compounds that exert localized anti-restenotic effects while minimizing vascular and cellular damage are essential for the long-term success of drug delivery stents.

However, significant progress has been made in the field of local, site specific, delivery of drugs via implantable medical devices. Drugs that once were systemic, typically administered orally, intravenously, or subcutaneously can now be delivered to a specific effected site by delivery from an implantable medical device. Drugs may be loaded into polymers coated onto implantable medical devices or may be bound to the medical device directly. Site specific therapy far exceeds the benefits of systematic delivery for many drugs. Therefore, compounds that exert localized effects while minimizing vascular and cellular damage are essential for the long-term success of drug delivery stents

### SUMMARY OF THE INVENTION

Described herein is an in situ drug delivery platform that can be used to locally administer beneficial levels of phosphodiesterase-5 inhibitors without systemic side effects. In one embodiment, the drug delivery platform is a medical device including, without limitations, stents, catheters, micro-particles, probes, and vascular grafts.

In one embodiment, a medical device is described comprising an implantable device for the site-specific, controlled delivery of a therapeutic amount of a phosphodiesterase-5 (PD-5) inhibitor. In another embodiment, the medical device the phosphodiesterase-5 (PD-5) inhibitor has a molecular structure selected from the group consisting of Formula 1,

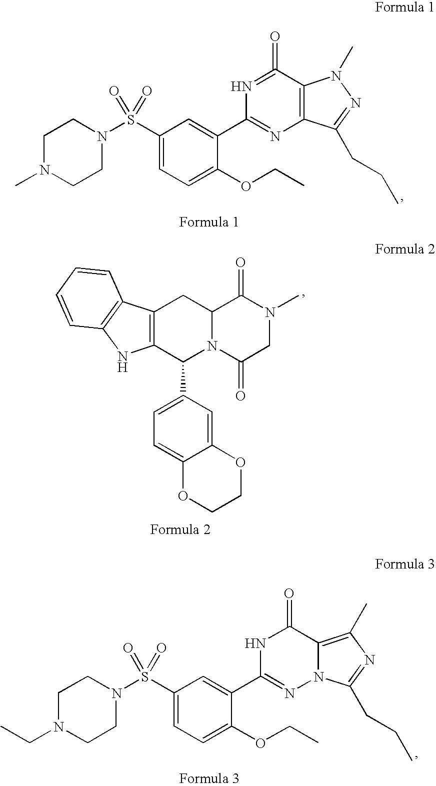

pharmaceutically acceptable derivatives, and combinations thereof.

In another embodiment, the phosphodiesterase-5 (PD-5) inhibitor is selected from the group consisting of 1-[4-ethoxy-3-(6,7-dihydro-1-methyl-7-oxo-3-propyl-1H-pyrazolo[4,3-d]pyrimidin-5-yl)phenylsulfonyl]-4-methylpiperazine citrate, (6R-trans)-6-(1,3-benzodioxol-5-yl)-2,3,6,7,12,12a-hexahydro-2-methyl-pyrazino[1′,2′:1,6]pyrido[3,4-b]indole-1,4-dione, 4-[2-ethoxy-5-(4-ethylpiperazin-1-yl)sulfonyl-phenyl]-9-methyl-7-propyl-3,5,6,8-tetrazabicyclo[4.3.0]nona-3,7,9-trien-2-one, pharmaceutically acceptable derivatives, and combinations thereof.

In one embodiment, the medical device is selected from the group consisting of stents, catheters, micro-particles, probes and vascular grafts. In anther embodiment the stent is a vascular stent, esophageal stent, urethral stent or biliary stent. In another embodiment, the vascular stent is provided with a coating comprising sildenafil, tadalafil, vardenafil, pharmaceutically acceptable derivatives, or combinations thereof.

In one embodiment, the coating further contains a biocompatible polymer. In another embodiment, the coating comprises between about 1 μg to about 1000 μg of phosphodiesterase-5 (PD-5) inhibitor and a polymer wherein said phosphodiesterase-5 (PD-5) inhibitor and said polymer are in a ratio relative to each other of approximately 1 part phosphodiesterase-5 (PD-5) inhibitor to approximately between 1 to 9 parts polymer.

In one embodiment, a method is described of increasing site specific concentrations of nitric oxide comprising providing a vascular stent having a coating comprising a phosphodiesterase-5 (PD-5) inhibitor; and implanting the vascular stent into a blood vessel lumen wherein the phosphodiesterase-5 (PD-5) inhibitor is released into tissue adjacent said blood vessel lumen; wherein the phosphodiesterase-5 (PD-5) inhibitor comprises sildenafil, tadalafil, vardenafil, pharmaceutically acceptable derivatives, or combinations thereof. In another embodiment, the coating comprises between about 1 μg to about 1000 μg of a phosphodiesterase-5 (PD-5) inhibitor and a polymer wherein said phosphodiesterase-5 (PD-5) inhibitor and said polymer are in a ratio relative to each other of approximately 1 part phosphodiesterase-5 (PD-5) inhibitor to approximately between 1 to 9 parts polymer.

In one embodiment, a method is described for producing a medical device comprising providing medical device to be coated; compounding sildenafil, tadalafil, vardenafil, pharmaceutically acceptable derivatives, or combinations thereof with a carrier compound; and coating the medical devices with the sildenafil, tadalafil, vardenafil, pharmaceutically acceptable derivatives, or combinations thereof compounded with said carrier compound. In another embodiment, the medical device is a vascular stent. In another embodiment, the carrier compound is a biocompatible polymer.

In one embodiment, a medical device is described comprising a stent having a coating comprising sildenafil, tadalafil, vardenafil, pharmaceutically acceptable derivatives, or combinations thereof; and at least one additional therapeutic agent selected from the group consisting of antiplatelet agents, antimigratory agent, antifibrotic agents, antiproliferatives, antiinflammatories and combinations thereof providing that the additional therapeutic agent. In another embodiment, the at least one additional therapeutic agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. In another embodiment, the at least one additional therapeutic agent comprises at least one compound selected from the group consisting of sirolimus (rapamycin), tacrolimus (FK506), everolimus (certican), temsirolimus (CCI-779) and zotarolimus (ABT-578).

In one embodiment, a method is described of treating or inhibiting restenosis comprising providing a vascular stent having a coating comprising sildenafil, tadalafil, vardenafil, pharmaceutically acceptable derivatives, or combinations thereof and at least one additional therapeutic agent selected from the group consisting of antiplatelet agents, antimigratory agent, antifibrotic agents, antiproliferatives, antiinflammatories and combinations thereof; and implanting the vascular stent into a blood vessel lumen wherein the sildenafil, tadalafil, vardenafil, pharmaceutically acceptable derivatives, or combinations thereof and at least one additional therapeutic agent are released into tissue adjacent to said blood vessel lumen.

### DEFINITION OF TERMS

Bioactive Agent: As used herein “bioactive agent” shall include any drug, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Bioactive agents can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, and other rapamycin hydroxyesters may be used in combination with the terpolymers.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein “biodegradable” refers to a polymeric composition that is biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however, all bioresorbable polymers are considered biodegradable. Biodegradable polymers are capable of being cleaved into biocompatible byproducts through chemical- or enzyme-catalyzed hydrolysis.

Nonbiodegradable: As used herein “nonbiodegradable” refers to a polymeric composition that is biocompatible and not subject to being broken down in vivo through the action of normal biochemical pathways.

Not Substantially Toxic: As used herein “not substantially toxic” shall mean systemic or localized toxicity wherein the benefit to the recipient is out-weighted by the physiologically harmful effects of the treatment as determined by physicians and pharmacologists having ordinary skill in the art of toxicity.

Pharmaceutically Acceptable: As used herein “pharmaceutically acceptable” refers to all derivatives and salts that are not substantially toxic at effective levels in vivo.

### DETAILED DESCRIPTION OF THE INVENTION

Nitric oxide (NO) has long been established as an effective vasodilator. When delivered in adequate concentration in a responsive vessel, the resulting chain of events will result in vasodilatation, inhibition of thrombosis formation and other related effects. The method of action of NO proceeds as follows. Endothelium-derived or exogenously generated NO activates soluble guanylate cyclase. Guanylate cyclase catalyzes the conversion of guanosine triphosphate (GTP) to 3′,5′-cyclic guanosine monophosphate (cGMP) and pyrophosphate. Therefore, activating guanylate cyclase with NO results in an increased concentration of cGMP in vascular smooth muscle cells (SMC). The increased concentrations of cGMP in SMC results in increased intracellular Ca2+, which causes muscle relaxation. Increased intracellular Ca⁺² concentrations result in smooth muscle relaxation which accounts for NO's vasodilating effects.

In scenarios where vessel damage limits the vessels ability to mediate a NO-response it is challenging to promote an increased ability to locally generate NO at the desired site of action, in order to attain normal vascular function and off-set or inhibit on-going vascular pathology. In such a case, the inventors have proposed the local delivery of a phosphodiesterase inhibitor from an implantable medical device.

Phosphodiesterase inhibitors function to inhibit the photodiesterase enzymes which function to degrade cGMP which in turn stifles SMC relaxation via deteriorating vasodilatation. The problem with using systemic phosphodiesterase inhibitors to treat vascular complications is that the amount of drug necessary for treatment would vastly increase the risk of undesired systemic side effects, to the point of being potentially toxic to the patient. As a non-limiting example, phosphodiesterase-5 inhibitors are extremely popular systemic drugs for treating erectile dysfunction (ED) in men.

Therefore, local, site specific delivery of phosphodiesterase inhibitors would improve the vascular platelet response to endogenous NO by extending the intracellular survival of cGMP in local cells, by inhibiting enzymes belonging to the phosphodiesterase family which rapidly degrade cGMP. Local delivery can also prolong the beneficial effects of NO within the treated vessel and minimize systemic exposure to the drug. The main benefits of local delivery of a phosphodiesterase would be comprised of increased intensity and duration of vasodiolatory response, increased vascular thromboresistance and inhibition of SMC proliferation.

In one embodiment, the phosphodiesterase inhibitors are specific to phosphodiesterase-5. In one embodiment, a phosphodiesterase-5 inhibitor is provided such as, but not limited to 1-[4-ethoxy-3-(6,7-dihydro-1-methyl-7-oxo-3-propyl-1H-pyrazolo[4,3-d]pyrimidin-5-yl) phenylsulfonyl]-4-methylpiperazine (sildenafil or Viagra®) as depicted in Formula 1.

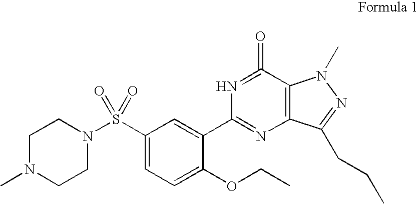

In another embodiment, a phosphodiesterase-5 inhibitor is provided such as, but not limited to (6R-trans)-6-(1,3-benzodioxol-5-yl)-2,3,6,7,12,12a-hexahydro-2-methyl-pyrazino[1′,2′:1,6]pyrido[3,4-b]indole-1,4-dione (tadalafil or Cialis®) as depicted in Formula 2.

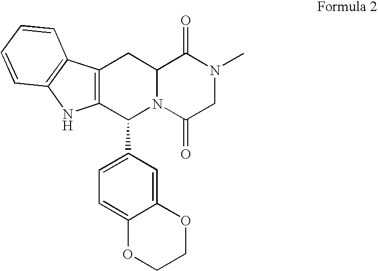

In yet another embodiment, a phosphodiesterase-5 inhibitor is provided such as, but not limited to 4-[2-ethoxy-5-(4-ethylpiperazin-1-yl)sulfonyl-phenyl]-9-methyl-7-propyl-3,5,6,8-tetrazabicyclo[4.3.0]nona-3,7,9-trien-2-one (vardenafil or Levitra®) as depicted in Formula 3.

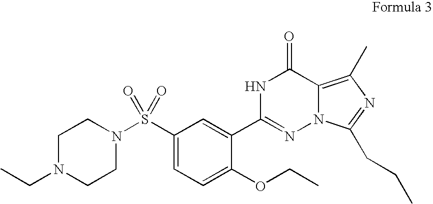

It will be understood by those skilled in the art, that Formula 1, 2 and 3 are but three of many pharmaceutically acceptable phosphodiesterase-5 inhibitors. Many other pharmaceutically acceptable forms can be synthesized. Moreover, many derivatives are also possible that do not affect the efficacy or mechanism of action of the phosphodiesterase-5 inhibitors. Therefore, 1-[4-ethoxy-3-(6,7-dihydro-1-methyl-7-oxo-3-propyl-1H-pyrazolo[4,3-d]pyrimidin-5-yl) phenylsulfonyl]-4-methylpiperazine (sildenafil or Viagra®), (6R-trans)-6-(1,3-benzodioxol-5-yl)-2,3,6,7,12,12a-hexahydro-2-methyl-pyrazino[1′,2′:1,6]pyrido[3,4-b]indole-1,4-dione (tadalafil or Cialis®), 4-[2-ethoxy-5-(4-ethylpiperazin-1-yl)sulfonyl-phenyl]-9-methyl-7-propyl-3,5,6,8-tetrazabicyclo[4.3.0]nona-3,7,9-trien-2-one (vardenafil or Levitra®) and pharmaceutically acceptable derivatives, salts and combinations thereof are all encompassed by the description herein.

The phosphodiseterase-5 inhibitors discussed herein may be added to implantable medical devices. The phosphodiesterase-5 inhibitors may be incorporated into the polymer coating applied to the surface of a medical device or may be incorporated into the polymer used to form the medical device. The phosphodiesterase-5 inhibitor may be coated to the surface with or without a polymer using methods including, but not limited to, precipitation, coacervation, and crystallization. The phosphodiesterase-5 inhibitor may be bound covalently, ionically, or through other intramolecular interactions, including without limitation, hydrogen bonding and van der Waals forces.

The medical devices used herein may be permanent medical implants, temporary implants, or removable devices. For example, and not intended as a limitation, the medical devices may include stents, catheters, micro-particles, probes, and vascular grafts.

In one embodiment, stents may be used as a drug delivery platform. The stents may be vascular stents, urethral stents, biliary stents, or stents intended for use in other ducts and organ lumens. Vascular stents, for example, may be used in peripheral, neurological, or coronary applications. The stents may be rigid expandable stents or pliable self-expanding stents. Any biocompatible material may be used to fabricate the stents, including, without limitation, metals and polymers. The stents may also be bioresorbable, In one embodiment, vascular stents are implanted into coronary arteries immediately following angioplasty.

In one embodiment, metallic vascular stents are coated with one or more phosphodiesterase-5 inhibitors, the compounds of Formula 1, Formula 2 and Formula 3. The phosphodiesterase-5 inhibitor may be dissolved or suspended in any carrier compound that provides a stable, un-reactive environment for the inhibitor. The stent can be coated with a phosphodiesterase-5 inhibitor coating according to any technique known to those skilled in the art of medical device manufacturing. Suitable non-limiting examples include impregnation, spraying, brushing, dipping and rolling. After the phosphodiesterase-5 inhibitor is applied to the stent, it is dried leaving behind a stable phosphodiesterase-5 inhibitor delivering medical device. Drying techniques include, but are not limited to, heated forced air, cooled forced air, vacuum drying or static evaporation. Moreover, the medical device, specifically a metallic vascular stent, can be fabricated having grooves or wells in its surface that serve as receptacles or reservoirs for the phosphodiesterase-5 inhibitors.

The effective amount of phosphodiesterase-5 inhibitor can be determined by a titration process. Titration is accomplished by preparing a series of stent sets. Each stent set will be coated, or contain different dosages of phosphodiesterase-5 inhibitor. The highest concentration used will be partially based on the known toxicology of the compound. The maximum amount of drug delivered by the stents will fall below known toxic levels. The dosage selected for further studies will be the minimum dose required to achieve the desired clinical outcome. The desired clinical outcome is defined as a site specific increase in NO concentration and associated effects.

In another embodiment, the phosphodiesterase-5 inhibitor is precipitated or crystallized on or within the stent. In yet another embodiment, the phosphodiesterase-5 inhibitor is mixed with a suitable biocompatible polymer (bioerodable, bioresorbable, or non-erodable). The polymer-phosphodiesterase-5 inhibitor blend can then be used to produce a medical device such as, but not limited to, stents, grafts, micro-particles, sutures and probes. Furthermore, the polymer-phosphodiesterase-5 inhibitor blend can be used to form controlled-release coatings for medical device surfaces. For example, and not intended as a limitation, the medical device can be immersed in the polymer-phosphodiesterase-5 inhibitor blend, the polymer-phosphodiesterase-5 inhibitor blend can be sprayed, or the polymer-phosphodiesterase-5 inhibitor blend can be brushed onto the medical device. In another embodiment, the polymer-phosphodiesterase-5 inhibitor blend can be used to fabricate fibers or strands that are embedded into the medical device or used to wrap the medical device.

In one embodiment, the polymer chosen must be a polymer that is biocompatible and minimizes irritation to the vessel wall when the medical device is implanted. The polymer may be either a biostable or a bioabsorbable polymer depending on the desired rate of release or the desired degree of polymer stability. Bioabsorbable polymers that could be used include poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid.

Also, biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

The polymer coatings or medical devices formed from polymeric material discussed herein may be designed with a specific dose of phosphodieserase-5 inhibitor suitable for the intended implantation site and intended duration of action. That dose may be a specific weight of inhibitor added or a phosphodiesterase-5 inhibitor to polymer ratio. In one embodiment, the medical device can be loaded with about 1 to about 1000 μg of phosphodiesterase-5 inhibitor; in another embodiment, about 5 μg to about 500 μg; in another embodiment about 10 μg to about 250 μg; in another embodiment, about 15 μg to about 150 μg. A ratio may also be established to describe how much phosphodiestrerase-5 inhibitor is added to the polymer that is coated to or formed into the medical device. In one embodiment a ratio of 1 part phosphodiesterase-5 inhibitor: 1 part polymer may be used; in another embodiment, 1:1-5; in another embodiment, 1:1-9; in another embodiment, 1:1-20.

In addition to the site specific delivery of phosphodiesterase-5 inhibitors, the implantable medical devices discussed herein can accommodate one or more additional bioactive agents. The choice of bioactive agent to incorporate, or how much to incorporate, will have a great deal to do with the polymer selected to coat or form the implantable medical device. A person skilled in the art will appreciate that hydrophobic agents prefer hydrophobic polymers and hydrophilic agents prefer hydrophilic polymers. Therefore, coatings and medical devices can be designed for agent or agent combinations with immediate release, sustained release or a combination of the two.

Exemplary, non limiting examples include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

Exemplary FKBP-12 binding agents include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers.

### EXAMPLES

### Providing a Metallic Surface with a Phosphodiesterase-5 Inhibitor-Eluting Coating

The following Examples are intended to illustrate a non-limiting process for coating metallic stents with a phosphodiesterase-5 inhibitor. One non-limiting example of a metallic stent suitable for use in accordance with the teachings described herein is the Medtronic/AVE S670™ 316L stainless steel coronary stent.

### Example 1

### Metal Stent Cleaning Procedure

Stainless steel stents were placed a glass beaker and covered with reagent grade or better hexane. The beaker containing the hexane immersed stents was then placed into an ultrasonic water bath and treated for 15 minutes at a frequency of between approximately 25 to 50 KHz. Next the stents were removed from the hexane and the hexane was discarded. The stents were then immersed in reagent grade or better 2-propanol and vessel containing the stents and the 2-propanol was treated in an ultrasonic water bath as before. Following cleaning the stents with organic solvents, they were thoroughly washed with distilled water and thereafter immersed in 1.0 N sodium hydroxide solution and treated at in an ultrasonic water bath as before. Finally, the stents were removed from the sodium hydroxide, thoroughly rinsed in distilled water and then dried in a vacuum oven over night at 40° C. After cooling the dried stents to room temperature in a desiccated environment they were weighed their weights were recorded.

### Example 2

### Coating a Clean, Dried Stent Using a Drug/Polymer System

In the following Example, ethanol is chosen as the solvent of choice. The phosphodiesterase-5 inhibitor is 1-[4-ethoxy-3-(6,7-dihydro-1-methyl-7-oxo-3-propyl-1H-pyrazolo[4,3-d]pyrimidin-5-yl)phenylsulfonyl]-4-methylpiperazine (sildenafil or Viagra®), herein referred to as sildenafil. Both the polymer and sildenafil are freely soluble ion ethanol. Persons having ordinary skill in the art of polymer chemistry can easily pair the appropriate solvent system to the polymer-drug combination and achieve optimum results with no more than routine experimentation.

250 mg of sildenafil is carefully weighed and added to a small neck glass bottle containing 2.8 ml of ethanol. The sildenafil-ethanol suspension is then thoroughly mixed until a clear solution is achieved.

Next 250 mg of polycaprolactone (PCL) is added to the sildenafil-ethanol solution and mixed until the PCL dissolved forming a drug/polymer solution.

The cleaned, dried stents are coated using either spraying techniques or dipped into the drug/polymer solution. The stents are coated as necessary to achieve a final coating weight of between approximately 10 μg to 1 mg. Finally, the coated stents are dried in a vacuum oven at 50° C. over night. The dried, coated stents are weighed and the weights recorded.

The concentration of drug loaded onto (into) the stents is determined based on the final coating weight. Final coating weight is calculated by subtracting the stent's pre-coating weight from the weight of the dried, coated stent.

### Example 3

### Coating a Clean, Dried Stent Using a Sandwich-Type Coating

A cleaned, dry stent is first coated with polyvinyl pyrrolidone (PVP) or another suitable polymer followed by a coating of sildenafil. Finally, a second coating of PVP is provided to seal the stent thus creating a PVP-sildenafil-PVP sandwich coated stent.

The Sandwich Coating Procedure:

100 mg of PVP is added to a 50 mL Erlenmeyer containing 12.5 ml of ethanol. The flask was carefully mixed until all of the PVP is dissolved. In a separate clean, dry Erlenmeyer flask 250 mg of sildenafil is added to 11 mL of ethanol and mixed until dissolved.

A clean, dried stent is then sprayed with PVP until a smooth confluent polymer layer was achieved. The stent was then dried in a vacuum oven at 50° C. for 30 minutes.

Next, successive layers of sildenafil are applied to the polymer-coated stent. The stent is allowed to dry between each of the successive sildenafil coats. After the final sildenafil coating has dried, three successive coats of PVP are applied to the stent followed by drying the coated stent in a vacuum oven at 50° C. over night. The dried, coated stent is weighed and its weight recorded.

The concentration of drug in the drug/polymer solution and the final amount of drug loaded onto the stent determine the final coating weight. Final coating weight is calculated by subtracting the stent's pre-coating weight from the weight of the dried, coated stent.

### Example 4

Coating a Clean, Dried Stent with Pure Drug

g of sildenafil is carefully weighed and added to a small neck glass bottle containing 12 ml of ethanol. The sildenafil-ethanol suspension is then heated at 50° C. for 15 minutes and then mixed until the sildenafil is completely dissolved.

Next a clean, dried stent is mounted over the balloon portion of angioplasty balloon catheter assembly. The stent is then sprayed with, or in an alternative embodiment, dipped into, the sildenafil-ethanol solution. The coated stent is dried in a vacuum oven at 50° C. over night. The dried, coated stent was weighed and its weight recorded.

The concentration of drug loaded onto (into) the stents is determined based on the final coating weight. Final coating weight is calculated by subtracting the stent's pre-coating weight from the weight of the dried, coated stent.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A medical device comprising an implantable device for the site-specific, controlled delivery of a therapeutic amount of a phosphodiesterase-5 (PD-5) inhibitor.

2. The medical device according to claim 1 wherein said phosphodiesterase-5 (PD-5) inhibitor has a molecular structure selected from the group consisting of Formula 1, 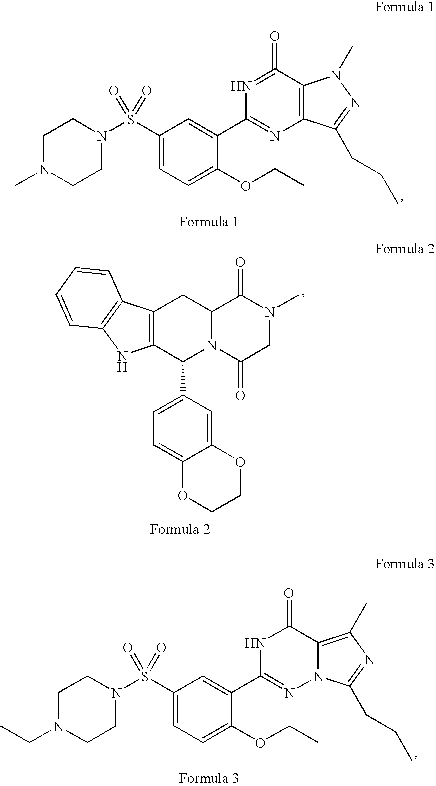
pharmaceutically acceptable derivatives, and combinations thereof.

3. The medical device according to claim 2 wherein said phosphodiesterase-5 (PD-5) inhibitor is selected from the group consisting of 1-[4-ethoxy-3-(6,7-dihydro-1-methyl -7-oxo-3-propyl-1H-pyrazolo[4,3-d]pyrimidin-5-yl)phenylsulfonyl]-4-methylpiperazine citrate, (6R-trans)-6-(1,3-benzodioxol-5-yl)-2,3,6,7,12,12a-hexahydro-2-methyl-pyrazino [1′,2′:1,6]pyrido[3,4-b]indole-1,4-dione, 4-[2-ethoxy-5-(4-ethylpiperazin-1-yl)sulfonyl-phenyl]-9-methyl-7-propyl-3,5,6,8-tetrazabicyclo[4.3.0]nona-3,7,9-trien-2-one, pharmaceutically acceptable derivatives, and combinations thereof.

4. The medical device according to any of claims 2 or 3 wherein said medical device is selected from the group consisting of stents, catheters, micro-particles, probes and vascular grafts.

5. The medical device according to claim 4 wherein said stent is a vascular stent, esophageal stent, urethral stent or biliary stent.

6. The medical device according to claim 5 wherein said vascular stent is provided with a coating comprising sildenafil, tadalafil, vardenafil, pharmaceutically acceptable derivatives, or combinations thereof.

7. The medical device according to claim 6 wherein said coating further contains a biocompatible polymer.

8. The medical device according to claim 7 wherein said coating comprises
between about 1 μg to about 1000 μg of phosphodiesterase-5 (PD-5) inhibitor and a polymer wherein said phosphodiesterase-5 (PD-5) inhibitor and said polymer are in a ratio relative to each other of approximately 1 part phosphodiesterase-5 (PD-5) inhibitor to approximately between 1 to 9 parts polymer.

9. A method of increasing site specific concentrations of nitric oxide comprising:
providing a vascular stent having a coating comprising a phosphodiesterase-5 (PD-5) inhibitor; and
implanting said vascular stent into a blood vessel lumen wherein said phosphodiesterase-5 (PD-5) inhibitor is released into tissue adjacent said blood vessel lumen;
wherein said phosphodiesterase-5 (PD-5) inhibitor comprises sildenafil, tadalafil, vardenafil, pharmaceutically acceptable derivatives, or combinations thereof.

10. The method according to claim 9 wherein said coating comprises:
between about 1 μg to about 1000 μg of a phosphodiesterase-5 (PD-5) inhibitor and a polymer wherein said phosphodiesterase-5 (PD-5) inhibitor and said polymer are in a ratio relative to each other of approximately 1 part phosphodiesterase-5 (PD-5) inhibitor to approximately between 1 to 9 parts polymer.

11. A method for producing a medical device comprising:
providing medical device to be coated;
compounding sildenafil, tadalafil, vardenafil, pharmaceutically acceptable derivatives, or combinations thereof with a carrier compound; and
coating said medical devices with said sildenafil, tadalafil, vardenafil, pharmaceutically acceptable derivatives, or combinations thereof compounded with said carrier compound.

12. The method according to claim 11 wherein said medical device is a vascular stent.

13. The method according to claim 11 further wherein said carrier compound is a biocompatible polymer.

14. A medical device comprising a stent having a coating comprising sildenafil, tadalafil, vardenafil, pharmaceutically acceptable derivatives, or combinations thereof; and
at least one additional therapeutic agent selected from the group consisting of antiplatelet agents, antimigratory agent, antifibrotic agents, antiproliferatives, antiinflammatories and combinations thereof providing that said additional therapeutic agent.

15. The medical device according to claim 14 wherein said at least one additional therapeutic agent is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

16. The medical device according to claim 14 wherein said at least one additional therapeutic agent comprises at least one compound selected from the group consisting of sirolimus (rapamycin), tacrolimus (FK506), everolimus (certican), temsirolimus (CCI-779) and zotarolimus (ABT-578).

17. A method of treating or inhibiting restenosis comprising:
providing a vascular stent having a coating comprising sildenafil, tadalafil, vardenafil, pharmaceutically acceptable derivatives, or combinations thereof and at least one additional therapeutic agent selected from the group consisting of antiplatelet agents, antimigratory agent, antifibrotic agents, antiproliferatives, antiinflammatories and combinations thereof; and
implanting said vascular stent into a blood vessel lumen wherein said sildenafil, tadalafil, vardenafil, pharmaceutically acceptable derivatives, or combinations thereof and at least one additional therapeutic agent are released into tissue adjacent to said blood vessel lumen.
